(19) United States
(12) Patent Application Publication
(10) Pub. No.: US 2006/0121114 A1
(43) Pub. Date: Jun. 8, 2006

(54) Method of manufacturing sustained release microbeads containing venlafaxine HCL

(76) Inventors: Amit Krishna Antarkar, Nagpur (IN); Poonam Prakash Vardam, Thane (West) (IN); Maya Janak Shah, Mumbai (IN); Rajendra Ghanshamial Lala, Mumbai (IN)

Correspondence Address: WINSTON & STRAWN LLP, 1700 K STREET, N.W., WASHINGTON, DC, 20006, US

(21) Appl. No.: 11/149,527

(22) Filed: Jun. 10, 2005

Related U.S. Application Data
(63) Continuation-in-part of application No. 11/138,913, filed on May 27, 2005.
Application No. 11/138,913 is a continuation of application No. PCT/IB03/05194, filed on Nov. 17, 2003.

(30) Foreign Application Priority Data
Nov. 28, 2002 (IN) 1054/MUM/2002

(51) Int. Cl. A61K 9/26

(52) U.S. Cl. 424/469

(57) ABSTRACT

A sustained release Venlafaxine composition that includes a plurality of non-agglomerated, uniformly-shaped and sized microbeads of inert core particles having a first coating layer. The first coating layer includes an active agent of Venlafaxine or a pharmaceutical acceptable salt thereof, a binder, and an anti-tack agent. The active agent is present in the first coating layer in a concentration of at least about 5% to about 70% by weight of the composition, the binder is present in an amount of at least 35% by weight of the active agent, or in a further layer located upon or below the first coating layer, or as an alternating layer between plural first layers, wherein the binder is present in an amount of less than about 2.5% by weight of the composition, and the anti-tack agent is present in the first coating layer in a concentration of about 2.5% to about 20% by weight based on the weight of the active agent. The composition is also substantially free of organic acid.

### CROSS-REFERENCE TO RELATED APPLICATIONS

The present application is a continuation-in-part of U.S. application Ser. No. 11/138,913, filed May 27, 2005, which is a continuation of International Application PCT/IB2003/005194, filed Nov. 17, 2003, the entire content of each of which is expressly incorporated herein by reference.

### FIELD OF THE INVENTION

The present invention relates to stable sustained release compositions, and methods of manufacturing thereof, that include Venlafaxine or its pharmaceutically acceptable salt for once a day dosing.

### BACKGROUND OF THE INVENTION

Venlafaxine HCl is an anti-depressant agent that is commonly recommended for a variety of diseases and disorders including, for example, manic disorder, attention deficit disorder, Parkinson's disease, and epilepsy. The recommended daily dosage for adults typically ranges form 75 mg to 350 mg per day, which is usually taken over the course of two to three doses per day. Since multiple dosing is inconvenient to patients, it is desirable to minimize the dosage frequency by tailoring a sustained release composition that is specific to a drug, based on desired pharmacokinetic and pharmacodynamic activity.

Venlafaxine HCl is highly water-soluble and has a potential problem of dose dumping and burst effect when using a controlled release matrix. Hence, a matrix delivery system is not suitable for consistent and prolonged delivery of Venlafaxine HCl to the site of action. It is thus desirable to develop dosage forms of Venlafaxine HCl to ensure consistent delivery and prolonged plasma levels, with insignificant contribution to the initial release in case of a failure of the system, thereby avoiding dose dumping.

Several methods of depositing Venlafaxine HCl on inert cores are generally known in the prior art. These cores are typically further coated with one or more polymeric layers to overcome the problems associated with matrix delivery systems.

U.S. Application Publication No. 2004/0131677 describes a programmed release composition including 10% to 80% Venlafaxine HCl by weight. Micronized Venlafaxine HCl is deposited on an inert core using a PVP alcoholic solution in a coating pan to obtain microgranules. The microgranules are coated with talc using the PVP solution and further coated with a plasticized ethylcellulose solution. The yield is not more than 92% by weight. This process requires periodically powdering the product with talc to diminish the static load, thereby interrupting the continuity of process and making it unsuitable for industrial application. The microgranules that are obtained are also not of adequate strength as the mechanical conditions in the fluid bed processor during the coating process causes rupturing of some of the microgranules, which further reduces the yield of the process.

PCT Publication WO 03/041692 is directed to extended release compositions that include Venlafaxine HCl in a concentration of 30% to 60% by weight. The Venlafaxine HCl is coated with a binder having a concentration of 0.5% to 10% by weight on an inert core. This coated core is then coated with an isolating layer and then further coated with polymer layer. The process utilizes water, ethanol, or a combination thereof, as a solvent mixture for spraying the Venlafaxine HCl. The process of utilizing water for spraying Venlafaxine HCl as described therein results, however, in the settling of product mass in a product container, thereby interrupting the continuity of the process. The process of utilizing ethanol as described therein is not sufficient in dissolving Venlafaxine HCl and the Venlafaxine HCl suspension in ethanol, when sprayed on an inert core utilizing PVP as a binder in a concentration of 0.5% to 10% by weight, will result in improper fluidization or changes in fluidization patterns during the process. This leads to inefficient loading of Venlafaxine HCl on inert seeds and results in drug loss and low batch yield, which is generally not more than 95% by weight.

PCT Publication WO 00/71099 describes a multiparticulate controlled release formulation of selective serotonin reuptake inhibitor (SSRI), such as fluvoxamine. The process includes deposition of SSRI, an organic acid and a polymeric material on an inert core to obtain a drug-loaded bead. These are coated with a rate controlling membrane, ammonio-methacrylate co-polymer, dibutyl sebacate and talc. However, the use of an organic acid with Venlafaxine HCl is usually not advisable. Moreover, organic acids may influence the physiochemical properties of the rate controlling membrane, thereby affecting the stability of such a formulation.

Venlafaxine HCl is more soluble in water as compared to its solubility in alcohol and thus water is a solvent of preferred choice. Such an approach would substantially reduce the processing time and cost, making the process more commercially viable. However, it is known that Venlafaxine HCl develops tack and static charge when depositing it on inert seeds by powder layering using an aqueous binder solution. This tendency of developing tackiness and static charge increases when Venlafaxine HCl is sprayed from an aqueous or hydroalcoholic binder solution or a dispersion on inert seeds. This problem of tackiness and static charge leads to further processing problems such as, for example, agglomeration of drug coated seeds and improper fluidization or changes in fluidization patterns during the process, leading to inefficient loading of Venlafaxine HCl on inert seeds and resulting in drug loss and low batch yields, and settling of the product mass in the product container, thereby interrupting the continuity of the process. The above-mentioned problems are especially common when equipment, such as a fluid bed bottom spray processor or coating pan, is used.

Thus, there is a need for improvements to the prior art methods of manufacture and the resulting pharmaceutical compositions, which are now provided by the present invention.

### SUMMARY OF THE INVENTION

The present invention relates to a sustained release Venlafaxine composition that includes a plurality of non-agglomerated, uniformly-shaped and sized microbeads of inert core particles having a first coating layer including an active agent of Venlafaxine or a pharmaceutical acceptable salt thereof, a binder, an anti-tack agent, and any other suitable excipients. The active agent is present in the first coating layer in a concentration of at least about 5% to about 70% by weight of the composition, the binder is present in the first coating layer in a concentration of at least about 35% by weight of the active agent, or in a further layer located upon or below the first coating layer, or as an alternating layer between plural first layers, wherein the binder is preferably present in an amount of less than about 2.5% by weight of the composition, and the anti-tack agent is present in the first coating layer at a concentration of about 2.5% to about 20% by weight based on the weight of the active agent. Additionally, the composition is substantially free of organic acid.

Preferably, the inert core particles include sugar spheres that optionally include starch or microcrystalline cellulose and have a particle size of about 300 microns to about 1680 microns. The anti-tack agent is preferably talc, colloidal silicon dioxide, magnesium stearate, glyceryl behenate, glyceryl monostearate, or a combination thereof and is at least about 10.5% by weight of the active agent.

In one embodiment, the sustained release composition further includes one or more additives of an excipient, binder, polymer or plasticizer present either in the first coating layer or in a further layer of the composition with the additive(s) being present in an amount sufficient to enhance the sustained release properties of the composition, and with the composition exhibiting a pH-independent in-vitro release of the active agent.

Preferably, the inert excipient is starch, lactose, microcrystalline cellulose, low viscosity grade hydroxypropylcellulose, mannitol, pulverized sugar, sorbitol, or combinations thereof and is present in an amount of between about 2% and about 12% by weight of the active agent. The inert excipient is also preferably present either in the first coating layer or in a further layer located upon the first coating layer.

Preferably, the binder of the sustained release composition is selected from hydroxypropylmethylcellulose, hydroxypropylcellulose, polyvinylpyrollidone, sugar, acrylic acid, methacrylic acid copolymer, or a combination thereof and is present either in the first coating layer, wherein the binder is present in an amount of at least about 35% by weight of the active agent, or in a further layer located upon or below the first coating layer, or as an alternating layer between plural first layers, wherein the binder is preferably present in an amount of less than about 2.5% by weight of the active agent.

In another embodiment, the sustained release composition further includes a second coating layer upon the first coating layer, which includes a non-functional polymer and anti-tack agent, and third coating layer upon the second coating layer, which includes a functional polymer and plasticizer.

Preferably, the non-functional polymer is selected from hydroxypropylmethylcellulose, hydroxypropylcellulose, polyvinylpyrollidone, sugar, acrylic acid, methacrylic acid copolymer, or a combination thereof and is present in an amount of up to 5% by weight of the core and first layer. Preferably, the functional polymer is ethylcellulose hydroxypropylmethylcellulose, methacrylic acid copolymer, or a combination thereof in an amount of between 1% and 25% by weight of the core and first layer. Preferably, the plasticizer is a hydrophilic or hydrophobic plasticizer present in an amount of about 5% to 25% by weight of the functional polymer.

In another embodiment, the sustained release composition has a pH-independent release rate of Venlafaxine HCl from the composition at the end of 1, 4, 8 and 10 hours that lies in the range of not more than about 15%, about 30% to about 50%, about 55% to about 80%, and not less than about 65%, respectively, when measured in-vitro in a USP type II apparatus at about 100 rpm in about 900 ml of any one of distilled water, 0.1 N HCl, pH 4.5 acetate buffer, pH 6.8 phosphate buffer, or pH 7.2 phosphate buffer at 37° C.

The present invention also relates to a method for manufacturing a sustained release Venlafaxine composition that includes preparing non-agglomerated, uniformly-shaped and sized microbeads by providing a first coating layer upon each of a plurality of inert core particles. The first coating layer includes an active agent of Venlafaxine or a pharmaceutical acceptable salt thereof, a binder, an anti-tack agent, and any other suitable excipients. The resulting microbeads are essentially free of organic acid and exhibit a pH-independent in-vitro release of the active agent with essentially no latent period.

Preferably, the active agent is present in the first coating layer in a concentration of at least about 5% to about 70% by weight of the composition, the binder is present in the first coating in a concentration of less than about 2.5% by weight based on the weight of the composition, and the anti-tack agent is present in the first coating layer at a concentration of about 2.5% to about 20% by weight based on the weight of the active agent. The first coating layer is deposited upon the inert cores by forming an admixture of the active agent, the anti-tack agent, optionally with an inert excipient such as starch using aqueous solution of the binder. The solution is sprayed onto the particles and first layer is deposited thereon, which are dried to a final water content of less than 3% by weight.

Another embodiment preferably includes depositing the active agent, anti-tack agent and binder, present in a concentration of at least about 35% by weight of the active agent, on the inert cores. The anti-tack agent in the first coating layer is preferably in a concentration of at least about 10.5% by weight of the active agent.

The inert excipient is preferably starch, lactose, microcrystalline cellulose, low viscosity grade hydroxypropylcellulose, mannitol, pulverized sugar, sorbitol, or combinations thereof. The excipient is present in the microbeads in an amount of between about 2% and about 12% by weight of the active agent, and the inert excipient is preferably present either in the first coating layer or in a further layer located upon the first coating layer. Preferably, the spraying is carried out in a fluid bed bottom spray processor at an inlet air temperature of between about 50° C. and about 80° C., an outlet air temperature of between about 40° C. and about 55° C., an atomization air pressure of between about 0.8 bars and about 3.5 bars, and a fluidization flap open between about 15% and about 90%.

In another embodiment, the method further includes coating the drug cores preferably with a second aqueous medium as a single second layer or alternating second layers with plural first layers. The second aqueous media preferably includes a non-functional polymer and anti-tack agent. Preferably, the method also includes coating the first and second layers with a third layer, which includes a functional polymer and a plasticizer.

In one embodiment, the solution preferably includes water, methanol, ethanol, isopropanol, or a combination thereof, and is sprayed onto the particles as a single first layer or as an alternating first layer with a binder therebetween to form drug cores.

In another embodiment, the coating of the cores is preferably carried out in fluid bed bottom spray processor at an inlet air temperature of between about 20° C. and about 60° C., an outlet air temperature of between about 20° C. and about 45° C., an atomization air pressure of between about 0.8 bars and about 3.5 bars, and a fluidization flap open of between about 15% and about 90%.

In another embodiment, the yield of the process is preferably at least about 95% by weight, and the method further includes filling the sustained release microbeads into capsules of size 5 to size 10 for delivering a dose of up to about 150 mg of active gent for once a day dosing.

### DETAILED DESCRIPTION OF THE PREFERRED EMBODIMENTS

Surprisingly, it has been found that the above-mentioned problems are improved upon when Venlafaxine HCl solution is sprayed on inert seeds with a binder in a concentration of at least about 35% by weight of Venlafaxine HCl, to provide less than about 2.5% by weight of Venlafaxine HCl in the composition, and with an anti-tack agent in a concentration of at least about 10.5% Venlafaxine HCl by weight in the solution, or at least about 0.5% by weight of the composition. Preferably, the binder is hydroxypropylmethylcellulose (HPMC) and the anti-tack agent is talc. It has also been found that depositing Venlafaxine HCl on inert seeds with an anti-tack agent, such as talc, and an inert excipient, such as starch, and using a binder, preferably in a concentration ranging from a minimum amount that is sufficient to assist in adhering the drug particles to the drug core to a maximum amount of less than about 2.5% by weight of the composition, can also improve upon problems associated with prior art methods of processing Venlafaxine HCl. Advantageously, the yields associated with the preferred embodiments of the present invention are preferably not less than about 95% and more preferably not less than 97% by weight.

The present invention thus provides a viable, continuous method for manufacturing of stable, sustained release pharmaceutical composition that preferably includes between about 5% and about 70% Venlafaxine by weight of the composition or its pharmaceutical acceptable salt. More preferably, the composition includes between about 10% and about 60% Venlafaxine by weight of the composition. Preferably, the composition of Venlafaxine HCl is manufactured for once a day dosing, wherein Venlafaxine HCl is processed in an aqueous media and the composition is substantially free, and preferably completely free, of organic acid.

The term “substantially free of organic acid” means that the organic acid is present, if at all, in a concentration that does not deleteriously affect the stability of the composition. Typically, no organic acid would be included at all in the composition but very small amounts could be tolerated if included, provided that the composition remains stable throughout the shelf life of the product and complies with all the pharmacoepial conditions and standards.

In a preferred embodiment of the invention, the sustained release pharmaceutical composition is manufactured in an aqueous medium in the form of agglomeration-free, uniformly-shaped and sized microbeads of adequate strength. The yield of the manufacturing process of the present invention is preferably at least about 95% by weight, and more preferably at least about 97% by weight.

The sustained release composition is preferably capable of being filled into a capsule for delivery of a therapeutically effective amount of Venlafaxine. Preferably, the sustained release composition is capable of being filled into a capsule of between size 0 and 5, and more preferably of size 5 for easy administration to a patient.

In the preferred embodiment, the formulation of the sustained release composition includes a therapeutically effective amount of Venlafaxine. Preferably, the sustained release formulation includes Venlafaxine HCl, a binder, and an anti-tack agent. The composition also preferably includes an inert excipient that is layered or coated on inert seeds in an aqueous media. Additionally, the composition is further preferably coated with an anti-tack agent and a non-functional polymer, and then coated with a functional polymer and a plasticizer.

In another embodiment, the sustained release composition includes an alternate layer of an admixture of Venlafaxine HCl, an inert excipient and an anti-tack agent with binder, preferably HPMC, on inert seeds in an aqueous media. The layer is then coated with an anti-tack agent and a non-functional polymer, and further coated with a functional polymer and a plasticizer.

Unlike the process disclosed in U.S. Application Publication No. 2004/0131677, however, the methods of the present invention advantageously do not require powdering the composition with talc to diminish the static load thereof. Also, the methods of the present invention result in a higher yield that is preferably at least about 95% by weight and more preferably at least about 97% by weight. The methods also advantageously do not cause rupturing of microbeads during the coating process, which indicates that the resulting microbeads are of adequate strength.

In another embodiment of the invention, the sustained release composition includes inert seeds coated with Venlafaxine HCl, an anti-tack agent, and a binder that is preferably HPMC in an aqueous media. The inert seeds are further coated with a functional polymer and a plasticizer. This embodiment is structurally different from the disclosure in PCT publication WO 03/041692 as it does not require coating of the drug cores with an isolating, protecting, or separating layer.

The present invention is also related to a process for the preparation of a sustained release composition that includes Venlafaxine or its pharmaceutically acceptable salt, preferably Venlafaxine HCl, on inert seeds wherein Venlafaxine HCl is processed in an aqueous media. The preferred embodiment preferably includes two sequential stages: preparation of the drug core and preparation of the sustained release composition.

The drug core is preferably prepared by mixing Venlafaxine HCl, an anti-tack agent and, optionally, an inert excipient to obtain an admixture, which is deposited on inert seeds, such as sugar spheres, using an aqueous binder solution. In one embodiment of the invention, Venlafaxine HCl is in an admixture with an anti-tack agent and an inert excipient, and the admixture is deposited on a sugar sphere as an alternating layer with a binder solution in water. In another embodiment, a suspension includes Venlafaxine HCl, a binder and an anti-tack agent in water where Venlafaxine HCl is in a dissolved state and the suspension is deposited as a single layer on a sugar sphere to obtain the drug core.

Preferably, the drug core is then hardened and dried using equipment such as a coating pan, a tray drier or a fluid bed drier or their likes to achieve a moisture content of less than about 5% by weight. Preferably, the moisture content is less than about 3% by weight, and more preferably, less than about 2% by weight. After drying, the drug core is optionally coated with a layer of a non-functional polymer and an anti-tack agent to obtain a hardened drug core. In another embodiment of the invention, a non-functional polymer and an anti-tack agent are deposited from an aqueous media as a single layer on the drug core. Alternatively, a non-functional polymer in water is deposited with an anti-tack agent as an alternate layer on the drug core to obtain the hardened drug core.

The inert seeds used to form the drug cores are preferably sugar spheres that include sugar and starch. Alternatively, the inert seeds include microcrystalline cellulose or any other suitable inert material. The particle size of the sugar sphere is preferably in the range of about 300 microns to 1680 microns, and more preferably about 500 microns to 1200 microns.

The anti-tack agent is selected from a group that preferably includes talc, colloidal silicon dioxide, magnesium stearate, glyceryl behenate, glyceryl monostearate, or a combination thereof. In the preferred embodiment, the anti-tack agent is talc with or without colloidal silicon dioxide. Preferably, the anti-tack agent is in a concentration from between about 2.5% and about 20% by weight of Venlafaxine HCl to provide between about 0.1% to about 14% by weight of the composition. More preferably, the anti-tack agent is present in a concentration from between about 4% and about 15% by weight of Venlafaxine HCL, and between about 2% and about 10.5% by weight of the composition.

Inert excipients are selected from a group that preferably includes starch, lactose, microcrystalline cellulose, low viscosity grade hydroxypropylcellulose, mannitol, pulverized sugar, sorbitol and their likes, and may be used alone or in combination with other excipients. More preferably, the inert excipient is starch and is used in a concentration from between about 2% and about 12% by weight of Venlafaxine HCl to provide between about 0.1% and about 8.5% by weight of the composition. Even more preferably, the concentration of the inert excipient is between about 3% and about 8% by weight of Venlafaxine HCl and between about 1.5% and 5.5% by weight of the composition.

The binder is selected from a group that preferably includes HPMC, hydroxypropylcellulose, polyvinylpyrollidone, sugar, acrylic acid and methacrylic acid copolymer and may be used alone or in combination with other binders. More preferably, the binder is HPMC used in a concentration of between about 1% and about 55% by weight of Venlafaxine HCl, and between about 0.05% and about 38% by weight of the composition. More preferably, the binder is present in a concentration of between about 1.5% and about 35% by weight of Venlafaxine HCl and between about 0.08% and about 20% by weight of the composition. The non-functional polymer is selected from a group that preferably includes HPMC, hydroxypropylcellulose, polyvinylpyrollidone, sugar, acrylic acid and methacrylic acid copolymer and is used in a concentration of between about 0.1% and about 5% by weight of the drug core. The HPMC that is used as a binder and as a non-functional polymer preferably has a nominal viscosity of about 3 cps to about 15 cps when measured as 2% solution in water at about 20° C.

In the preferred embodiment, the solvent for depositing Venlafaxine HCl on the inert seeds is water, however, other solvents such as alcohol, hydroalcoholic mixtures, organic solvents or a combination thereof can also be used.

The process described herein is consistent with the fluidization pattern when Venlafaxine HCl is sprayed in the aqueous media, preferably in a fluid bed bottom spray coater that enables efficient deposition of Venlafaxine HCl on inert seeds and thereby providing a high yield. In contrast to the process disclosed in PCT publication WO 00/71099, the process of the present invention does not require use of organic acid for tailoring drug release characteristic.

The sustained release composition is preferably prepared by coating the drug core or hardened drug core with a combination of a functional polymer, a plasticizer and optionally an anti-tack agent. Preferably, the functional polymer used for coating the drug core or the hardened drug core is selected from a group that includes methacrylic acid copolymer, cellulose derivatives preferably alkyl cellulose, such as ethylcellulose hydroxypropylmethylcellulose, and can be used alone or in mixtures thereof or in combination with other polymers. Preferably, the functional polymer is in a concentration of between about 1% and about 25% by weight of the drug core or the hardened drug core, and more preferably, the functional polymer is ethylcellulose. A ready-to-use aqueous dispersion of ethylcellulose may also be used as a functional polymer for the preparation of the sustained release composition. The ethylcellulose used as a functional polymer preferably has a nominal viscosity of about 9 cps to about 11 cps when measured as a 5% by weight solution in toluene:alcohol (80:20) at about 25° C.

The plasticizer used in the sustained release composition is selected from a group that preferably includes hydrophobic and hydrophilic plasticizers. Preferably, the plasticizer is triacetin and triethylcitrate and is used in a concentration of about 5% to about 25%, and more preferably in a concentration of about 10% to about 20%, by weight of the functional polymer.

Optionally, an anti-tack agent, such as talc, may also be added to the coating of the drug core or the hardened drug core when the aqueous dispersion includes a functional polymer. Preferably, the anti-tack agent is present in a concentration between about 1% and about 30% by weight of the functional polymer, and more preferably between about 5% and about 20% by weight of the functional polymer.

The resulting sustained release composition exhibits a pH-independent release profile at least for a period of about 10 hours when analyzed in-vitro using USP type II (paddle) dissolution testing apparatus at 100 rpm in 900 ml in various media at different pHs at 37° C. In addition, the composition exhibits essentially no latent period.

The process described herein may be carried out completely or in part in an aqueous or a non-aqueous media such as methanol, ethanol, isopropanol or a combination thereof. Additionally, the process can be carried out in equipment such as a fluid bed bottom spray processor, a coating pan and their likes, and can be carried out using a single equipment or a combination of more than one equipment.

### EXAMPLES

The present invention for the preparation of sustained release microbeads comprising Venlafaxine HCl is illustrated by the following Examples that are merely for the purpose of illustration and are not to be regarded as limiting the scope of the invention or the manner in which it can be practiced.

### Examples 1 to 3

Venlafaxine HCl was passed through a 200 mesh ASTM and mixed with starch and talc in a planetary mixer for about 10 minutes. HPMC E05 was dispersed and dissolved in water. The concentration of HPMC in water can be up to about 10% by weight. Sugar spheres were loaded in a coating pan and the HPMC solution was sprayed on the sugar spheres. When the desired level of wetting was observed, the admixture of Venlafaxine HCl, starch and talc was layered until the wetted agglomerated sugar spheres were unagglomerated. This operation was repeated until the total quantity of the admixture was applied. Thereafter, the drug cores were dried in tray drier. The drug cores were then sieved through a desired mesh and checked for moisture content and particle size. The drug cores of undesirable size (utilizable residue) that were retained above and below the desired mesh were mixed with water and added to a non-functional polymer suspension containing talc. The suspension was filtered through an appropriate mesh and was sprayed on the drug cores in a coating pan to obtain hardened drug cores. The solid content of this suspension in water may be up to about 20% by weight. These hardened drug cores were dried in tray drier and checked for moisture content and particle size.

To prepare the sustained release microbeads, ethylcellulose was dispersed and dissolved in a mixture of methanol and methylene chloride (in a ratio of 2:3) and triacetin was added to the solution. The solution was filtered through an appropriate mesh and was sprayed on the hardened drug cores in a fluid bed bottom spray processor to obtain the sustained release microbeads. Other organic solvents such as isopropanol or acetone can also be used. Methanol and methylene chloride may be used in a ratio of between about 1:9 to about 9:1, and preferably in a ratio of about 2:3. An aqueous ready-to-use dispersion of ethylcellulose can also be used. Preferably, the solid content of the dispersion or solution should not be more than about 20% by weight, and more preferably not more than 15% by weight.

The processing parameters during the coating process were adjusted to achieve an inlet air temperature of about 20° C. to about 60° C., and preferably of about 30° C. to about 45° C., an outlet air temperature of about 20° C. to about 45° C., and preferably of about 25° C. to about 40° C., an atomization air pressure of about 0.8 bars to about 3.5 bars, and a fluidization flap open from about 15% to about 90%, and preferably between about 25% and about 70%. The sustained release microbeads were dried in the same equipment maintaining the inlet temperature between about 50° C. to about 80° C. and outlet temperature between about 40° C. to about 60° C. to achieve a moisture content of less than about 5%, preferably less than about 3%, and more preferably less than about 2% by weight. Alternatively the coating may also be carried out in a coating pan.

| | | | |
|---|---|---|---|
| | % by weight | % by weight | % by weight |
| Ingredients | Example 1 | Example 2 | Example 3 |

I) Drug Core

| | | | |
|---|---|---|---|
| Venlafaxine HCl | 16.60 | 33.39 | 56.89 |
| Sugar sphere | 66.42 | 48.25 | 22.71 |
| Starch | 1.66 | 0.83 | 2.00 |
| Talc | 2.49 | 1.67 | 3.00 |
| HPMC E05 | 0.33 | 0.42 | 1.50 |

Hardened Drug Core

| | | | |
|---|---|---|---|
| HPMC E05 | 1.31 | 1.33 | 1.72 |
| Talc | 0.39 | 0.40 | 0.52 |

II) Sustained Release Microbeads

| | | | |
|---|---|---|---|
| Ethyl Cellulose | 9.82 | 12.47 | 10.60 |
| Triacetin | 0.98 | 1.24 | 1.06 |
| Total | 100 | 100 | 100 |
| Particle size of | 18-20 # ASTM | 20-22 # ASTM | 25-30 # ASTM |
| sugar sphere | | | |

### Example 4

HPMC E05 was dispersed and dissolved in water and Venlafaxine HCl was dissolved in water. The solutions were mixed and talc was added. The mixed solution was filtered through an appropriate mesh and was sprayed on sugar spheres in a fluid bed bottom spray processor with an inlet air temperature between about 50° C. and about 80° C., an outlet air temperature about 40° C. and about 55° C., an atomization air pressure of between about 0.8 bars and about 3.5 bars, and a fluidization flap open between about 15% and about 90%. After spraying the drug suspension, the drug cores were dried in the same equipment maintaining the inlet temperature between about 50° C. and about 80° C. and the outlet temperature between about 40° C. and about 60° C. to achieve a moisture content of less than about 5%, preferably less than about 3%, and more preferably less than about 2% by weight. The total solid content in the spray suspension was up to about 30% by weight.

The process of coating the drug cores after drying in fluid bed bottom spray processor was continued as described in Examples 1 to 3 to obtain the sustained release microbeads comprising of Venlafaxine HCl. The yield of the process was not less than about 95% by weight and generally greater than about 97% by weight.

| | |
|---|---|
| Ingredients | % by weight |

I) Drug Core

| | |
|---|---|
| Venlafaxine HCl | 39.22 |
| Sugar sphere (18-20 # ASTM) | 25.37 |
| Talc | 5.88 |
| HPMC E05 | 19.61 |

II) Sustained Release Microbeads

| | |
|---|---|
| Ethyl Cellulose | 9.01 |
| Triacetin | 0.91 |
| Total | 100 |

The sustained release microbeads comprising Venlafaxine HCl prepared in various strength as illustrated above are capable of being filled in capsules of various size including, for example, size 0 to size 5 and for various dosages including, for example, 37.5 mg, 75 mg and 150 mg of Venlafaxine. Preferably, the sustained release microbeads are capable of being filled in the smallest capsules of size 5 for the dose of 37.5 mg of Venlafaxine for ease of administration and patient acceptance.

Dissolution Studies:

The performance of the sustained release microbeads were monitored by in vitro dissolution testing using USP type II (paddle) apparatus at 100 rpm in 900 ml distilled water/0.1 N HCl/pH 4.5 acetate buffer/pH 6.8 phosphate buffer/pH 7.2 phosphate buffer at 37° C. The acceptance criteria for any batch of sustained release microbeads is provided below:

| Time (hours) | Cumulative % drug release |
|---|---|
| 1 | NMT 15% |
| 4 | 30% to 50% |
| 8 | 55% to 80% |
| 10 | NLT 65% |

If a batch of the sustained release microbeads releases the drug too slowly to comply with the dissolution release profile of the formulation, a portion of the drug cores or of the lower coating level may be added to comply with the above-mentioned drug release profile. In contrast, if a batch of the sustained release microbeads releases the drug too rapidly, then it may receive an additional coating to comply with the desired drug release profile. The sustained release microbeads provide a pH independent in-vitro release of Venlafaxine HCl at least for a period of 10 hours without any latent period, and more preferably for a period of at least 15 hours. The present invention thus provides a method for manufacturing sustained release microbeads of Venlafaxine HCl without the problems of dose dumping and burst effect.

Bioequivalence Studies:

A randomized two way, two period, two treatment cross over bioequivalence study of the Venlafaxine HCl sustained release capsules comprising 150 mg of Venlafaxine HCl (test) was compared with 2×75 mg extended release capsules (reference) comprising 75 mg of Venlafaxine HCl in 12 healthy male, adult human volunteers under fasting conditions. The results were as follows.

| Pharmacokinetic parameters | Test | | Reference | |
|---|---|---|---|---|
| Cmax (ng/ml) | 161.75 | (±12.27) | 162.00 | (±17.61) |
| (mean ± std. dev.) | | | | |
| Tmax (hrs.) | 3.08 | (±0.35) | 3.16 | (±0.32) |
| (mean ± std. dev.) | | | | |
| AUC (0-30) ng · hr/ml | 1539.40 | (±249.80) | 1565.83 | (±238.02) |
| (mean ± std. dev.) | | | | |

The sustained release microbeads of Venlafaxine HCl are bio-equivalent and provide therapeutic blood levels of the Venlafaxine HCl for once a day dosing of therapeutically effective amounts of Venlafaxine HCl. Additionally, the sustained release microbeads comprising of therapeutic effective amount Venlafaxine HCl are stable at least for a period of 2 years.

The term “about,” as used herein, should generally be understood to refer to both the corresponding number and a range of numbers. Moreover, all numerical ranges herein should be understood to include each whole integer within the range.

While illustrative embodiments of the invention are disclosed herein, it will be appreciated that numerous modifications and other embodiments can be devised by those of ordinary skill in the art. Features of the embodiments described herein can be combined, separated, interchanged, and/or rearranged to generate other embodiments. Therefore, it will be understood that the appended claims are intended to cover all such modifications and embodiments that come within the spirit and scope of the present invention.

What is claimed is:

1. A sustained release Venlafaxine composition, comprising a plurality of non-agglomerated, uniformly-shaped and sized microbeads of inert core particles having a first coating layer comprising an active agent of Venlafaxine or a pharmaceutical acceptable salt thereof, a binder, and an anti-tack agent, wherein the active agent is present in the first coating layer in a concentration of at least about 5% to about 70% by weight of the composition, the binder is present in an amount of at least 35% by weight of the active agent, or in a further layer located upon or below the first coating layer, or as an alternating layer between plural first layers, wherein the binder is present in an amount of less than about 2.5% by weight of the composition, and the anti-tack agent is present in the first coating layer in a concentration of about 2.5% to about 20% by weight based on the weight of the active agent, wherein the composition is substantially free of organic acid.

2. The sustained release composition of claim 1, wherein the inert core particles comprise sugar spheres that optionally include starch or microcrystalline cellulose and have a particle size of about 300 microns to about 1680 microns.

3. The sustained release composition of claim 1, wherein the anti-tack agent is talc, colloidal silicon dioxide, magnesium stearate, glyceryl behenate, glyceryl monostearate, or a combination thereof and is at least about 10.5% by weight of the active agent.

4. The sustained release composition of claim 1, further comprising one or more additives of an excipient, binder, polymer or plasticizer present either in the first coating layer or in a further layer of the composition with the additive(s) being present in an amount sufficient to enhance the sustained release properties of the composition, and wherein the composition exhibits a pH-independent in-vitro release of the active agent.

5. The sustained release composition of claim 4, wherein the inert excipient is starch, lactose, microcrystalline cellulose, low viscosity grade hydroxypropylcellulose, mannitol, pulverized sugar, sorbitol, or combinations thereof and is present in an amount of between about 2% and about 12% by weight of the active agent, wherein the inert excipient is present either in the first coating layer or in a further layer located upon the first coating layer.

6. The sustained release composition of claim 1, wherein the binder is selected from hydroxypropylmethylcellulose, hydroxypropylcellulose, polyvinylpyrollidone, sugar, acrylic acid, methacrylic acid copolymer, or a combination thereof and is present either in the first coating layer, wherein the binder is present in an amount of at least about 35% by weight of the active agent, or in a further layer located upon or below the first coating layer, or as an alternating layer between plural first layers, wherein the binder is present in an amount of less than about 2.5% by weight of the composition.

7. The sustained release composition of claim 4, which further comprises a second coating layer upon the first coating layer comprising a non-functional polymer and anti-tack agent; and third coating layer upon the second coating layer and comprising a functional polymer and plasticizer.

8. The sustained release composition of claim 7, wherein the non-functional polymer is selected from hydroxypropylmethylcellulose, hydroxypropylcellulose, polyvinylpyrollidone, sugar, acrylic acid, methacrylic acid copolymer, or a combination thereof and is present in an amount of up to 5% by weight of the inert core particles and the first layer; the functional polymer is ethylcellulose hydroxypropylmethylcellulose, methacrylic acid copolymer, or a combination thereof in an amount of between 1% and 25% by weight of the inert core particles and the first layer; and the plasticizer is a hydrophilic or hydrophobic plasticizer present in an amount of about 5% to 25% by weight of the functional polymer.

9. The sustained release composition of claim 8, wherein the pH independent release rate of Venlafaxine HCl from the composition at the end of 1, 4, 8 and 10 hours lies in the range of not more than about 15%, about 30% to about 50%, about 55% to about 80%, and not less than about 65%, respectively, when measured in-vitro in a USP type II apparatus at about 100 rpm in about 900 ml of any one of distilled water, 0.1 N HCl, pH 4.5 acetate buffer, pH 6.8 phosphate buffer, or pH 7.2 phosphate buffer at 37° C.

10. A method for manufacturing a sustained release Venlafaxine composition, which comprises preparing non-agglomerated, uniformly-shaped and sized microbeads by providing a first coating layer upon each of a plurality of inert core particles, the first coating layer comprising an active agent of Venlafaxine or a pharmaceutical acceptable salt thereof, a binder, and an anti-tack agent, with the microbeads being essentially free of organic acid and exhibiting pH-independent in-vitro release of the active agent with essentially no latent period.

11. The method of claim 10, wherein the active agent is present in the first coating layer in a concentration of at least about 5% to about 70% by weight of the composition, the binder is present in an amount of at least about 35% by weight of the active agent, the anti-tack agent is present in the first coating layer at a concentration of about 2.5% to about 20% by weight based on the weight of the active agent, and the first coating layer is deposited upon the inert core particles by forming an admixture solution of the active agent, the binder and the anti-tack agent and, optionally with an inert excipient in an aqueous solution, followed by spraying the solution onto the particles and drying the particles to a final water content of less than 3% by weight.

12. The method of claim 11, wherein the inert excipient is present in the solution and is starch, lactose, microcrystalline cellulose, low viscosity grade hydroxypropylcellulose, mannitol, pulverized sugar, sorbitol, or combinations thereof and is present in the microbeads in an amount of between about 2% and about 12% by weight of the active agent, wherein the inert excipient is present either in the first coating layer or in a further layer located upon the first coating layer.

13. The method of claim 11, wherein the spraying is carried out in a fluid bed bottom spray processor at an inlet air temperature of between about 50° C. and about 80° C., an outlet air temperature of between about 40° C. and about 55° C., an atomization air pressure of between about 0.8 bars and about 3.5 bars, and a fluidization flap open between about 15% and about 90%.

14. The method of claim 11, wherein the solution includes water, methanol, ethanol, isopropanol, or a combination thereof, and is sprayed onto the inert core particles as a single first layer to form drug cores.

15. The method of claim 11, wherein the binder is selected from hydroxypropylmethylcellulose, hydroxypropylcellulose, polyvinylpyrollidone, sugar, acrylic acid, methacrylic acid copolymer, or a combination thereof and is present in the microbeads in an amount of less than about 2.5% by weight of the active agent.

16. The method of claim 11, wherein coating of the inert core particles is carried out in fluid bed bottom spray processor at an inlet air temperature of between about 20° C. and about 60° C., an outlet air temperature of between about 20° C. and about 45° C., an atomization air pressure of between about 0.8 bars and about 3.5 bars, and a fluidization flap open of between about 15% and about 90%.

17. The method of claim 11, which further comprises coating the drug cores with a second aqueous medium as a single second layer, wherein the second aqueous media comprises a non-functional polymer and anti-tack agent.

18. The method of claim 11, which further comprises coating the first or second layers with a third layer comprising a functional polymer and a plasticizer.

19. The method of claim 17, wherein the non-functional polymer is selected from hydroxypropylmethylcellulose, hydroxypropylcellulose, polyvinylpyrollidone, sugar, acrylic acid, methacrylic acid copolymer, or a combination thereof and is present in an amount of up to 5% by weight of the inert core particles and the first layer; the functional polymer is ethylcellulose hydroxypropylmethylcellulose, methacrylic acid copolymer, or a combination thereof in an amount of between 1% and 25% by weight of the inert core particles and the first layer; and the plasticizer is a hydrophilic or hydrophobic plasticizer present in an amount of about 5% to 25% by weight of the functional polymer.

20. The method of claim 11, wherein the yield of the process is at least about 95% by weight, and which further comprises filling the sustained release microbeads into capsules of size 5 to size 10 for delivering a dose of up to about 150 mg of active gent for once a day dosing.

21. The method of claim 11, wherein the pH independent release rate of Venlafaxine HCl from the composition at the end of 1, 4, 8 and 10 hours lies in the range of not more than about 15%, about 30% to about 50%, about 55% to about 80% and not less than about 65%, respectively, when measured in-vitro in a USP type II apparatus at about 100 rpm in about 900 ml of any one of distilled water, 0.1 N HCl, pH 4.5 acetate buffer, pH 6.8 phosphate buffer, or pH 7.2 phosphate buffer at 37° C.
